(19) United States
(12) Patent Application Publication
(10) Pub. No.: US 2023/0197198 A1
(43) Pub. Date: Jun. 22, 2023

(54) SYSTEMS AND METHODS FOR DETECTING GENOME EDITS

(71) Applicant: MONSANTO TECHNOLOGY LLC, Saint Louis, MO (US)

(72) Inventor: Yuanji ZHANG, Chesterfield, MO (US)

(21) Appl. No.: 17/924,948

(22) PCT Filed: May 12, 2021
(86) PCT No.: PCT/US2021/031957

§ 371 Date: Nov. 11, 2022

Related U.S. Application Data
(60) Provisional application No. 63/025,498, filed on May 15, 2020.

(51) Int. Cl. G16B 30/10

(52) U.S. Cl. CPC G16B 30/10

(57) ABSTRACT

Example systems and methods are provided for identifying genome edits in genomes based on patterns associated with joints in target sequences of the genomes. One example method includes receiving, by a computing device, a request to identify at least one edit in an output genome, where the output genome includes one or more edits to the genome and where a reference sequence is representative of an unedited version of the genome. The method also includes identifying, by the computing device, the at least one edit of the one or more edits, based on sequence reads associated with the output genome mapped to the reference sequence relative to one or more reference edit patterns and reporting, by the computing device, the identified at least one edit.

### CROSS-REFERENCE TO RELATED APPLICATION

This application claims the benefit of, and priority to, U.S. Provisional Application No. 63/025,498, filed on May 15, 2020. The entire disclosure of the above-referenced application is incorporated herein by reference.

### FIELD

The present disclosure generally relates to systems and methods for use in detecting and/or identifying one or more genome edits, and in particular, to systems and methods for detecting and/or identifying genome edits based on reference patterns associated with target sites of genomes.

### BACKGROUND

This section provides background information related to the present disclosure which is not necessarily prior art.

Conventional breeding techniques for improving plant and animal stocks rely on controlled matings or crosses of parents, in which each parent conveys a given allele to produce at least one progeny organism comprising relevant alleles. Among organisms with diploid or polyploid genomes, production of a true-breeding stock with the desired combination of alleles generally requires all of the alleles included in the genome and the allele for each locus be found on both or all chromosome sets (for diploid and polyploid organisms, respectively). This may require many hundreds, thousands, or more crosses, depending on the number of traits that need to be introgressed into a given germplasm. In some instances, conventional breeding techniques cannot overcome certain genetic linkages to allow for stacking of desirable traits.

Deliberate and directable genome editing technologies, such as the clustered regularly interspersed short palindromic repeats (CRISPR) technology, are known to accelerate the process of introducing traits into a line, and of reducing the number of crosses that are necessary to generate a stable line with the desired traits. While CRISPR-based technologies can effectuate very precise and efficient editing of targeted nucleotide sequences, the genomic alterations (e.g., inversions, deletions, translocations, and insertions, etc.) induced by these technologies are not predictable. Therefore, there is a need for systems and methods for detecting, analyzing and characterizing genomic alterations induced by genome editing technologies.

### SUMMARY

This section provides a general summary of the disclosure, and is not a comprehensive disclosure of its full scope or all of its features.

Example embodiments of the present disclosure relate to methods for identifying edits in output genomes. In one example embodiment, such a method generally includes receiving, by a computing device, a request to identify at least one edit in an output genome, the output genome based on an input genome and one or more edits to the input genome; mapping, by the computing device, multiple sequence reads from the output genome onto one or more reference sequences, wherein the one or more reference sequences are representative of the input genome; identifying, by the computing device, from a data structure, the at least one edit based on one or more reference edit patterns matching a pattern associated with the at least one edit, wherein the one or more reference edit patters are defined by segments of the multiple sequence reads of the output genome mapped onto the one or more reference sequences; and reporting, by the computing device, the identified at least one edit in response to the request.

In another example embodiment, an example method of identifying one or more edits in an output genome to which one or more edits have been made generally includes identifying, by a computing device, the one or more edits in the output genome based on at least one reference pattern identified by mapping multiple sequence reads of the output genome onto a reference sequence of an unedited version of the genome.

In still another example embodiment, an example method of identifying one or more edits in an output genome to which one or more edits have been made generally includes receiving, by a computing device, a request to identify at least one edit in an edited genome, the edited genome based on an input wild type genome and one or more edits to the input wild type genome, wherein one or more reference sequences are representative of the input wild type genome; mapping, by the computing device, sequence reads from the edited genome to the one or more reference sequences; identifying, by the computing device, from a data structure, the at least one edit of the one or more edits, based on one or more reference edit patterns matching a pattern associated with the at least one edit; and reporting, by the computing device, the identified at least one edit in response to the request.

In still a further example embodiment, an example method for detecting one or more edits in an output genome to which one or more edits have been made generally includes receiving, by a computing device, a request to detect at least one edit in an output genome, the output genome based on an input genome and one or more edits to the input genome, wherein one or more reference sequences are representative of the input genome; detecting, by the computing device, the at least one edit of the one or more edits, based on sequence reads associated with the output genome mapped to the one or more reference sequences relative to one or more reference edit patterns; and reporting, by the computing device, the detected at least one edit.

In yet a further example embodiment, an example method for detecting one or more edits in an output genome to which one or more edits have been made generally includes receiving, by a computing device, a request to detect at least one edit in an edited genome, the edited genome based on an input wild type genome and one or more edits to the input wild type genome, wherein one or more reference sequence(s) is representative of the input wild type genome; detecting, by the computing device, the at least one edit of the one or more edits, based on sequence reads mapped to the one or more reference sequence(s), in terms of one or more reference edit patterns; and reporting, by the computing device, the detected at least one edit.

Example embodiments of the present disclosure also relate to systems for identifying edits in output genomes. In one example embodiment, such a system generally includes a computing device configured to receive a request to identify at least one edit in an output genome, the output genome based on an input genome and one or more edits to the input genome; map multiple sequence reads from the output genome onto one or more reference sequences, wherein the one or more reference sequences are representative of the input genome; identify, from a data structure, the at least one edit based on one or more reference edit patterns matching a pattern associated with the at least one edit, wherein the one or more reference edit patters are defined by segments of the multiple sequence reads of the output genome mapped onto the one or more reference sequences; and report the identified at least one edit in response to the request.

Example embodiments of the present disclosure also relate to non-transitory computer-readable storage media including executable instructions for identifying edits in output genomes. In one example embodiment such a non-transitory computer-readable storage medium includes instructions, which, when executed by at least one processor, cause the at least one processor to receive a request to identify at least one edit in an output genome, the output genome based on an input genome and one or more edits to the input genome; map multiple sequence reads from the output genome onto one or more reference sequences, wherein the one or more reference sequences are representative of the input genome; identify, from a data structure, the at least one edit based on one or more reference edit patterns matching a pattern associated with the at least one edit, wherein the one or more reference edit patterns are defined by segments of the multiple sequence reads of the output sequence mapped onto the one or more reference sequences; and report the identified at least one edit in response to the request.

Further areas of applicability will become apparent from the description provided herein. The description and specific examples in this summary are intended for purposes of illustration only and are not intended to limit the scope of the present disclosure.

### DRAWINGS

The drawings described herein are only for illustrative purposes of selected embodiments and are not intended to limit the scope of the present disclosure.

FIG. 1 is a block diagram of an example system of the present disclosure suitable for use in detecting and/or identifying edits to a sequence representative of a genome based on reference patterns associated with sequence reads of the genome;

FIG. 2 is an example diagram of short amplicon sequencing (SASi) that may be employed with regard to a sequence included in the system of FIG. 1;

FIG. 3 is an example diagram of long amplicon sequencing (LASi) that may be employed with regard to a sequence included in the system of FIG. 1;

FIG. 4 is a block diagram of a computing device that may be used in the system of FIG. 1;

FIG. 5 is an example method, suitable for use with the system of FIG. 1, for detecting and/or identifying edits to a sequence based on reference patterns associated with sequence reads thereof;

FIGS. 6A-6C include example diagrams relating to inversion edits to an input sequence, which may be detected and/or identified by the method of FIG. 5;

FIGS. 7A-7B include example diagrams relating to homolog fragment targeting (HFT) edits to an input sequence, which may be detected and/or identified by the example method of FIG. 5;

FIGS. 8A-8B include example diagrams of inversion and deletion edits to an input sequence, which may be detected and/or identified by the method of FIG. 5;

FIGS. 9A-9B include example diagrams of HFT and deletion edits to an input sequence, which may be detected and/or identified by the method of FIG. 5;

FIGS. 10A-10B include example diagrams of multiple inversion and deletion edits to an input sequence, which may be detected and/or identified by the method of FIG. 5;

FIGS. 11A-11B include example diagrams of multiple inversion edits to an input sequence, which may be detected and/or identified by the method of FIG. 5;

FIG. 12A-12B include example diagrams of HFT, inversion, and deletion edits to an input sequence, which may be detected and/or identified by the method of FIG. 5; and

FIGS. 13A-13B illustrate example diagrams of a trans-fragment targeting (TFT) edit to an input sequence, which may be detected and/or identified by the example method of FIG. 5.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

The practice of the embodiments described in this disclosure includes, unless otherwise indicated, utilization of conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

As used herein, the term “target site” refers to a nucleotide sequence against which a genome editing technology binds and/or exerts cleavage, nickase, recombinase or transposase activity. A target site may be genic or non-genic.

As used herein, the term “genome editing technique” or “genome editing technology” refers to any composition or method that can modify a nucleotide sequence in a site-specific manner. In the present disclosure genome editing techniques include the use of endonucleases, recombinases, transposases, helicases and any combination thereof.

Genome editing techniques, such as CRISPRs, Transcription activator-like effector nucleases (TALENs), and Zinc finger nucleases (ZFNs), introduce breaks at targeted locations in the DNA, which when repaired, can result in alterations such as deletions, insertions, translocations and inversions. Use of genome editing techniques can accelerate the process of introducing traits into genomes and even allow for the stacking of linked traits whose genomic loci are in repulsion. While genome editing techniques introduce DNA breaks at predicted locations within a genome, the alterations (genome edits) produced by those breaks are unpredictable. Post editing it may be difficult to detect and characterize the particular alterations in the resulting genome, especially where the alterations are complex and/or span regions larger than a typical sequence read.

Uniquely, the systems and methods herein provide for detecting (and/or identifying) and characterizing alterations in an edited genomic sequence, based on pattern-recognition at junctions in the genome, as compared to an un-edited reference sequence of the genome. In particular, after a genome is edited, the output genome is sequenced, whereby sequence reads (or sequencing reads) of the genome are generated. By determining locations and orientations of segments of the sequence reads in the reference sequence (if present), the sequence reads, especially at junctions mapped onto the reference sequence, provide one or more patterns. The patterns are generally specific to a type of edit or combination of edits to the sequence, whereby the patterns may be searched and/or compared to reference patterns to identify or detect the associated edit or edits. The detection (and/or identification) and characterization of edits based on reference patterns is suitable for identification of simple insertion and deletion edits, and beyond to inversion edits, translocation edits, and other complex edits, and combinations thereof, etc. In this manner, efficient recognition between the edited sequence and the reference sequence is permitted, whereby edits, if made, including even complex edits in combination (e.g., inversion plus deletion edits, etc.), may be detected (and/or identified) and characterized. As such, robust systems and methods are provided to detect simple and/or complex edits independent of variable such as the numbers of edit target sites in the genome (e.g., as defined by guide RNAs (gRNAs) or other editing technologies, etc.), unexpected genome break points resulting from large chewing-back or deletions, or numbers of genes/loci involved.

What's more, the systems and methods herein provide substantial flexibility over conventional detection techniques, in that detection is based on differences between the edited sequence(s) and the reference sequence(s), at the cut sites (or junction sites), as indicative of the edit(s) being made, or not, and the type of the edit(s) (e.g., regardless of what edits were intended or expected, etc.). Such a level of detection provides substantial improvement over conventional techniques (e.g., the UDiTaS edit detection technique, which pre-builds expected edits for reference; etc.), which often are only able to determine if an attempted edit was successful, or not.

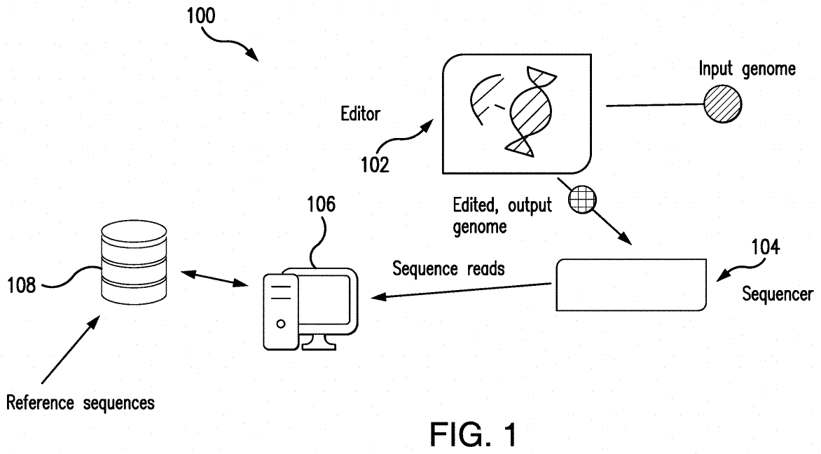

FIG. 1 illustrates an example system 100 in which one or more aspects of the present disclosure may be implemented. Although the system 100 is presented in one arrangement, other embodiments may include the parts of the system 100 (or additional parts) arranged otherwise depending on, for example, the manner in which genome edits are identified, selected, and/or edited into a genome sequence of an organism, the types and/or number of genome edits, etc.

In the example embodiment of FIG. 1, the system 100 generally includes a genome editor 102 (broadly, an editor) and a sequencer 104 associated with the genome editor 102. As will be described, the genome editor 102 is employed to perform various types of edits to an input genome, including, without limitation, insertion edits, deletion edits, inversion edits, translocation edits, or combinations thereof (e.g., by way of, through use of, through application of, etc. one or more editing technologies; etc.). And, the sequencer 104 is then employed to sequence the resulting edited, output genome, to provide one or more sequence reads of the genome (e.g., which may include 30 nucleotides, 50 nucleotides, 100 nucleotides, 150 nucleotides, 200 nucleotides, 250 nucleotides, 300 nucleotides, 350 nucleotides, 400 nucleotides, 450 nucleotides, 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1,000 nucleotides, 1,100 nucleotides, 1,150 nucleotides, 1,200 nucleotides, 1,250 nucleotides, 1,300 nucleotides, 1, 350 nucleotides, 1,400 nucleotides, 1,450 nucleotides, 1,500 nucleotides, 1,550 nucleotides, 1,600 nucleotides, 1,650 nucleotides, 1,700 nucleotides, 1,750 nucleotides, 1,800 nucleotides, 1,850 nucleotides, 1,900 nucleotides, 1,950 nucleotides, 2,000 nucleotides, or more or less, etc.). In connection therewith, the sequence reads of the genome, as provided by the sequencer 104, may be of the same length (e.g., 150 nucleotides (or base pairs), etc.).

Initially in the system 100, the genome editor 102 is configured to edit sequences of a genome, through one or more genome editing technologies, such as: CRISPR technology (e.g., CRISPR/Cas technology such as CRISPR/Cas9 technology, CRISPR/Cpf1 technology, etc.); zinc finger nuclease (ZFN) technology; transcription activator-like effector nucleases (TALEN®) technology; meganuclease technology; etc. With that said, the above technologies are provided as examples only and without limitation of the present disclosure with regard to editing sequences of a genome. The appropriate genome editing technology may readily be identified and executed by an ordinarily skilled artisan in connection with the genome editor 102, in accordance with the type and/or degree of editing of the genome required/desired, or more generally, the organism (e.g., plant, animal, etc.) selected and/or required.

More generally, as it pertains to the editing of a genome, an organism is initially nominated by a user, for example, to be edited. The organism may be, for example, a maize plant, a cotton plant, a canola plant, a soybean plant, a barley plant, a rye plant, a rice plant, a tomato plant, a wheat plant, an alfalfa plant, a sorghum plant, an Arabidopsis plant, a cucumber plant, a potato plant, a sweet potato plant, a pepper plant, a carrot plant, an apple plant, a banana plant, a pineapple plant, a blueberry plant, a blackberry plant, a raspberry plant, a strawberry plant, a cucurbit plant, a brassica plant, a citrus plant, an onion plant, etc. In some embodiments, the organism may be an animal, for example, a cow, a pig, a chicken, etc. Further, one or more genomic loci are nominated as the target for the edit, whereby, the genome editor 102 is configured to provide for one or more genome editing technologies (e.g., guide RNAs or gRNAs, etc.) to target the one or more loci for the edit.

In one example, the gRNA genome editing technology includes a sequence read matching to target locus, and sequence read(s) to form necessary secondary structures, and the target site includes a location in the target locus where gRNA matches to the sequence. And finally, the gRNA guides the edit enzyme to the target site in the locus for edit, where a cut site (or junction) is the location of the cut, which in this example, is a result of initial enzyme activity that will be followed by a number of steps in the cell to repair the cut. The gRNAs and edit enzymes are delivered into the target organism's cell nucleus for the edit, whereby the edit is made, or not, at the cut site (or junction) within the target site (and is enzyme-dependent).

In some embodiments, where the genome editing technology is a CRISPR nuclease complexed with one or more guide RNAs (gRNAs) that target the one or more loci for the edit. The gRNA includes a subsequence matching to target locus, and subsequence(s) to form necessary secondary structures which interact with the CRISPR nuclease. The gRNA guides the CRISPR nuclease to the target site in the target locus, where the CRISPR nuclease induces a cut site (or junction) is the location of the edit/alteration. If desired, a new or different DNA sequence can be inserted at the location cut by the Cas enzyme. Examples of CRISPR nuclease include Cas9, CasX, CasY, Cas12a (also known as Cpf1), Cas13a, Cas13b, Cas13d, Cas14a. In some embodiments, the CRISPR nuclease may be a synthetic or fused CRISPR nuclease (e.g., Fok1-fused dCas9, etc.), etc.

In some embodiments, the genome editing technology is zinc-finger nucleases (ZFNs). ZFNs are DNA binding proteins that induce double strand breaks of DNA at a user-specified location. The ZFN recognizes a specific DNA sequence and dimerizes around the target site. DNA cleavage only occurs when the ZFN has dimerized. The ZFN pair cuts the DNA strands and releases from the severed DNA. In some embodiments, a DNA sequence can be inserted into the genome as the TALEN-induced cleavage site through NHEJ or homology directed repair.

In some embodiments, the genome editing technology is a transcription activator-like effector nucleases (TALENs). TALENs are restriction enzymes designed to cut specific DNA sequences. The TALENs include a TAL-effector DNA-binding domain that recognizes and binds to a desired DNA sequence or genomic sequence of interest. A nuclease fused to the TAL-effector DNA-binding domain cuts the DNA by inducing double strand breaks. In some embodiments, a DNA sequence can be inserted into the genome as the TALEN-induced cleavage site through NHEJ or homology directed repair.

With continued reference to FIG. 1, once the desired edit is made (supposedly) by the genome editor 102 (e.g. through a genome editing technique such as the CRISPR/Cas system, through ZFNs, or through TALENs, etc.), the edited or output genome is sequenced, by the sequencer 104. Any suitable method for sequencing can be utilized to sequence the edited genome. Such methods may include, for example, but are not limited to, long-read sequencing methods (e.g. single molecule real time (SMRT) sequencing and nanopore DNA sequencing, etc.) and short-read sequencing methods (e.g. massively parallel signature sequencing (MPPS), polony sequencing, 454 pyrosequencing, illumina sequencing, combinatorial probe anchor sequencing (cPAS), sequencing by oligonucleotide ligation and detection (SOLiD) sequencing, ion torrent semiconductor sequencing, DNA nanoball sequencing, heliscope single molecule sequencing, and microfluidic systems, etc.). The precise sequencing method to be used by the sequencer 104 will depend on the size and nature of the genomic sequence to be assayed (and, potentially, on the organism selected/nominated for editing).

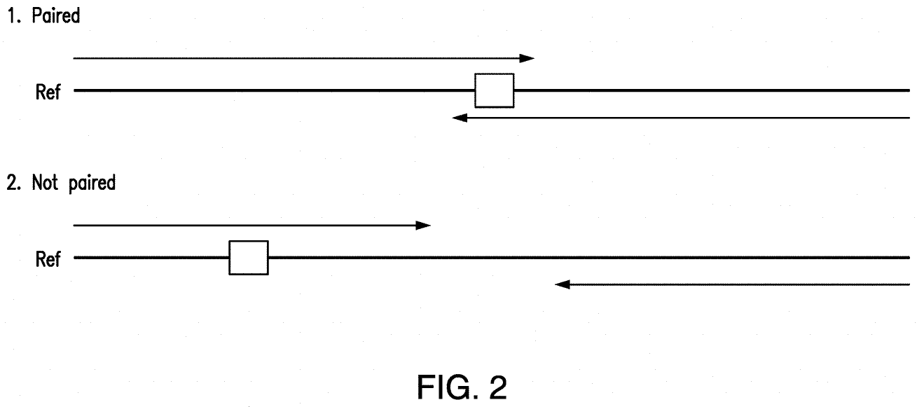

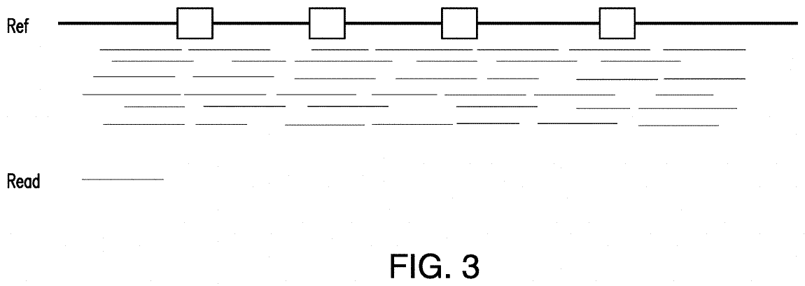

That said, in this example embodiment, the sequencer 104 may be configured to employ either short amplicon sequencing (SASi) or long amplicon sequencing (LASi). FIG. 2 schematically illustrates application of SASi by the sequencer 104 in connection with editing a desired genome (e.g., a reference sequence or “Ref” in this example, etc.), whereby numerous sequence reads (as represented by the arrowed lines) are read from an edited version of the genome, which includes one or more edits expected in target sites (denoted by the box). Specifically, for SASi, for example, the sequencer 104 is configured to sequence from both 5′ and 3′ ends of PCR products, resulting in Read1 (forward read, starting from forward primer at 5′ end) and Read2 (reverse read, starting from reverse primer at 3′ end), respectively. PCR primers are designed in such a way that the target site must be within Read1, Read2, or both (e.g., which may include, for example, up to 500 nucleotides each; etc.). When Read1 and Read2 overlap, this is paired SASi, otherwise it is un-paired SASi. As a result, a target site where an edit is initiated is covered by the sequence reads. Conversely, FIG. 3 schematically illustrates application of LASi by the sequencer 104 in connection with editing a desired genome (e.g., a reference sequence or “Ref” in this example, etc.), whereby numerous sequence reads (or “reads,” as represented by the discrete lines below the “Ref”) are generated from an edited version of the genome, which includes one or more edits (denoted by the four boxes) over an unedited version of the genome initiated from multiple target sites (denoted by the four boxes). Specifically, for LASi, for example, long PCR products are fragmented into smaller pieces, which are then sequenced by the sequencer 104 (and which are overlapping, as shown), whereby the fragmented pieces (sequence reads) may be used for sequencing library preparation. It is similar to genomic shotgun sequencing. As is shown, the LASi includes multiple sequence reads dispersed at different locations of the reference sequence. Regardless of the type, the sequencer 104 is configured to provide one or more sequence reads representative of the nucleotides making up the genome (and the locus targeted for editing, or not).

With reference again to FIG. 1, the system 100 also includes an edit detection computing device 106 (as part of an edit detection engine) and a data structure 108 coupled in communication thereto.

The computing device 106 is specifically configured by computer executable instructions to perform one or more of the operations of the edit detection engine, as described herein. In the illustrated embodiment, the computing device 106 and the data structure 108 are each shown as a standalone part of the system 100. However, in various other embodiments, it should be appreciated that the computing device 106 and/or the data structure 108 may be associated with, or incorporated with, other parts of the system 100, for example, the sequencer 104, etc. In addition, in one or more embodiments, the data structure 108 may be included at least in part in the computing device 106. Further, in various embodiments, the computing device 106 may be embodied as at least one physical computing device (e.g., server, etc.) and/or as a network or cloud service, via, for example, an application programming interface (API), or otherwise, etc.

In operation of the system 100, an input genome (e.g., an unedited genome, etc.), designated as a circle in FIG. 1, may be provided (e.g., based on the nominated organism, etc.) to the genome editor 102 along with a genome editing technology, which performs one or more desired edits to the input genome. As described above, the one or more edits to the input genome may include, without limitation, deletion edits, insertion edits, inversion edits, translocation edits, or combinations thereof, etc. In turn, the genome editor 102 is configured, as described above, to attempt the one or more desired edits, through the techniques described above and/or those known to skilled artisans. The genome editor 102 is configured to further provide an output, edited genome, which is then provided to the sequencer 104. And, the sequencer 104 is configured to then generate one or more sequence reads, for the output, edited genome, and to provide the one or more sequence reads to the computing device 106 (whereby in response the computing device 106 is configured to store the one or more sequence reads in the data structure 108 (e.g., as part of a reference sequence, etc.)).

It should be appreciated that, in this example embodiment, a reference sequence representative of the input genome is included in the data structure 108. This may be achieved in different manners. Specifically, for example, the reference sequence may be achieved as described above. Or, the reference sequence may be accessed from a public database of sequences for various organisms and stored in the data structure 108, or merely used by the computing device 106 (e.g., without separately storing the same in the data structure 108, etc.). Alternatively, the reference sequence may be provided to the computing device as part of other workflows or applications. Further, it should be appreciated that the input genome may be provided to the sequencer 104, whereby the sequencer 104 is configured to generate the reference sequence for the input genome (as a reference sequence), as one or more sequence reads, and provide the sequence to the computing device 106. The computing device 106 is configured, in turn, to store the generated sequence for the input genome in the data structure 108. Consistent with the above, where the reference sequence (or relevant part thereof) for the input genome is already available to the computing device 106 from other sources, sequencing of the input genome, by the sequencer 104, is omitted. In connection therewith, the reference sequence may be naturally occurring (or associated with a wild type (WT) genome), or it may be a mutant. In this way, multiple reference sequences may be accumulated, obtained, and/or stored in the data structure 108 for use herein.

In any case, once the reference sequences for multiple locus/loci of interest are obtained and/or stored in the data structure 108, the computing device 106 may receive a request to analyze one or more sequence reads for an output genome to determine or characterize edits thereto (e.g., to determine if appropriate edits were made to the genome, as a basis for selection of the genome for subsequent use, etc.). In connection therewith, the computing device 106 is configured to access the reference sequences, from the data structure 108, for example, and perform detection of the one or more edits included in the one or more sequence reads from the output genome (e.g., as received from the genome editor 102 and sequencer 104, etc.), relative to the reference sequences.

In this example embodiment, the computing device 106 is configured to employ an initial logic to determine potential edits in the output genome, prior to performing detection (although this is not required in all embodiments), based on a number of genes or loci and/or a number of target sites (e.g., as defined by guide RNAs (gRNAs) or other genome editing technology, etc.) employed by (or targeted by) the editor 102. In particular, the computing device 106 is configured to determine if any gene/locus is targeted by more than one editing technology, for example, more than one gRNA (e.g., determine the number of target sites in the output genome targeted for edit, etc.). In connection therewith, the computing device 106 is not actually detecting the gene/locus of a genome, but instead is detecting where sequence reads associated therewith have to be flipped or split, etc. to match a reference sequence for the unedited version of the genome (e.g., the computing device 106 is looking for where patterns of flips and spits in the sequence reads match that of a reference sequence, etc.). If the gene/locus is targeted by a single gRNA, for instance, the computing device 106 is configured to then perform detection for simple edits (e.g., insertion edits, deletion edits, etc.) on the output genome. Conversely, if the gene/locus is targeted by multiple gRNAs, the computing device 106 is configured to determine if the number of genes/locus is equal to one (e.g., determine if there is one gene associated with the edits of the output genome, etc.). And then, if the number of genes/locus is equal to one, the computing device 106 is configured to perform detection for simple, inversion, and homolog fragment targeting (HFT) edits on the output genome, at the target sites of the edits (e.g., as defined by the gRNA used for the edits, etc.). Otherwise, if the number is not equal to one, the computing device 106 is configured to perform detection for simple, inversion, HFT/TFT (trans-fragment targeting), and hairpin edits, at the target sites of the edits, at the target sites of the edits. It should be appreciated that in this way, by application of such initial logic, the potential edits included in the output genome may be limited based on the number of target sites, etc.

It should be appreciated that in order to detect the one or more edits, the computing device 106 is configured to map one or more of the sequence reads of the edited, output genome onto the reference sequence. For the sequence reads that span a junction of the edit (e.g., as associated with the target site, etc.) (or, potentially, are near thereto), the given sequence reads may be split into segments, whereby the location of the segments and the orientation of the segments define a pattern when mapped onto the reference sequence. The computing device 106 may then be configured to represent the pattern via one or more nomenclatures. In this example embodiment, the computing device 106 may express the sequence reads as R's and the target sites as T's along with U for upstream and D for downstream and an orientation indicator (e.g., “R,” etc.). So, for example, a sequence read may include two segments, with one upstream of a first target site, and the second upstream of a second target site as R1_T1U_T2U′. It should be appreciated that the nomenclature may be expanded, or not, to accurately, and reproducibly, represent the potential patterns provided by the mapping of the sequence reads onto the reference sequence.

The computing device 106 is then configured to detect the specific edits by searching for the pattern of the segments from the mapping in the data structure 108, which includes one or more reference patterns, defining particular types of edits by locations and orientations of segments of sequence reads, etc. The search may be limited by the specific edits (e.g., based on a number of target sites, a number of gRNAs, etc.) associated with the junctions, locations of the sequence reads (and/or segments), and/or orientations of the sequence reads (and/or segments within the sequence reads). The reference patterns may be predetermined and included in the data structure 108. The reference patterns (expressed according to the nomenclature above, or otherwise) may define, for example, missing segments of reference sequence reads as indicative of deletion edits, segments being separate but in the same orientation as indicative of insertion edits, segments being included in different sequences as indicative of translocation edits, etc. In another example, where the edit is an inversion edit, a reference pattern may define the inversion edit as a first junction including a first segment of a sequence read aligned to one location of the reference sequence, while a second segment of the same sequence read being inverted in orientation and aligned to another location spaced apart from the first segment (e.g., by the length of the inversion, etc.).

The reference patterns are included in the data structure 108, and described and referenced as appropriate (for comparison), as shown, for example, in Table 1. In particular, Table 1 includes three example reference patterns, which are segregated based on the number of target sites involved. The reference patterns included in the data structure 108 may be referred to as reference “read types,” which generally define locations and orientations of the sequence reads (and/or segments thereof) once mapped to the or reference sequence.

TABLE 1
Two segments of one sequence read mapped to one reference
sequence

| | |
|---|---|
| R1_T1U_T2U + | Segment 1 of a sequence read mapped |
| R1_T1D_T2D | to reference sequence 1, target site |
| | 1, upstream (R1T1U); and segment 2 |
| | of the same sequence read mapped to |
| | reference sequence 1, target site 2, |
| | upstream (R1T2U). The read type is |
| | ‘R1_T1U_T2U’ |
| | Segment 1 of another sequence read |
| | mapped to reference sequence 1, |
| | target site 1, downstream (R1T1D); |
| | and segment 2 of the same sequence |
| | read mapped to reference sequence 1, |
| | target site 2, downstream (R1T2D). |
| | The read type is R1_T1D_T2D. |
| | The two read types together define |
| | an edit pattern for an inversion |
| | edit between target sites 1 and 2. |
| . . . | . . . |

Two segments of one sequence read mapped to two separate reference
sequences

| | |
|---|---|
| R1T1U_R2T1D + | Segment 1 of a sequence read mapped |
| R1T1D_R2T2U. | to reference sequence 1, target site |
| | 1, upstream (R1T1U); and segment 2 |
| | of the same sequence read mapped to |
| | reference sequence 2, target site 1, |
| | downstream (R2T1D). The read type is |
| | ‘R1T1U_R2T1D’ |
| | Segment 1 of another sequence read |
| | mapped to reference sequence 2, |
| | target site 2, upstream (R2T2U); |
| | and segment 2 of the same sequence |
| | read mapped to reference sequence 1, |
| | target site 1, downstream (R1T1D). |
| | The read type is R1T1D_R2T2U. |
| | The two read types then define an |
| | edit pattern for a translocation |
| | edit where a piece from reference |
| | sequence 2 between target sites 1 |
| | and 2 is inserted into reference |
| | sequence 1, target site 1. |
| . . . | . . . |

It should be appreciated that the reference patterns included in Table 1 are provided for example only. Additional reference patterns, or read types, for each type of possible edit, and combinations thereof, may be compiled and stored in the data structure 108, etc.

As such, using the available reference patterns, the computing device 106 is thus configured to detect one or more edits, alone or in combination, in the output, edited genome as defined by the sequence reads and segments thereof. For example, where the pattern provided by the mapping of the sequence reads onto the reference sequence includes R1_T1U_T2U+R1_T1D_T2D, the search will identify the reference pattern of the first example from Table 1, whereby an inversion edit between target sites 1 and 2 is detected, identified, characterized, etc.

Further in the system 100, the computing device 106 may be configured to verify the detected edit(s) to one or more desired edits (e.g., confirm that the desired edits were actually made to the input genome, etc.) and to publish a report or otherwise notify one or more users of a verification result. The report for a given output genome (relative to a reference sequence) may include a description or characterization of the edit, including, for example, that locus A, from coordinate a to coordinate b, is inverted, etc., or more specifically, the report may include an encoded edit, such as, for example, S5d10, which indicates a deletion (as a type) starting from nucleotide 5 (as a starting coordinate at a target site) and extending for 10 nucleotides (as a length). The report may be published by displaying the report to a user associated with the computing device 106, or by transmitting the report, via email, SMS message, network-based application, etc., to a user associated with the system 100 (e.g., an initiator of the detection by system 100, etc.).

In some embodiments, the computing device 106 may be configured to assemble the sequence reads from the output genome back into a subsequence of the genome, and then map the assembled output sequence against the reference sequences in the data structure 104.

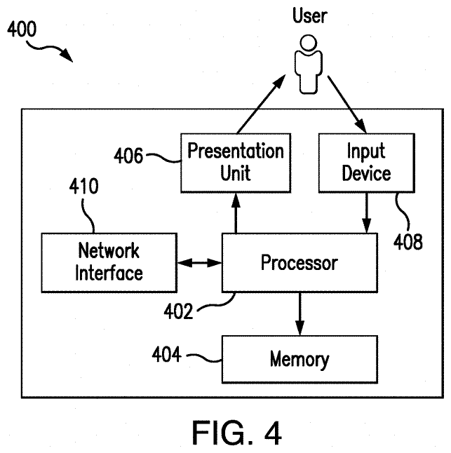

FIG. 4 illustrates an example computing device 400 that can be used in the system 100. The computing device 400 may include, for example, one or more servers, workstations, personal computers, laptops, tablets, smartphones, virtual devices, etc. In addition, the computing device 400 may include a single computing device, or it may include multiple computing devices located in close proximity or distributed over a geographic region, so long as the computing devices are specifically configured to operate as described herein. In the example embodiment of FIG. 1, at the least, and the sequencer 104 may include and/or may be implemented in one or more computing devices consistent with computing device 400. In addition, the genome editor 102 may be associated with and/or in communication with a computing device consistent with computing device 400. Also, in the example embodiment, the computing device 106 (or edit detection engine) and the data structure 108 may be understood to be computing devices, at least partially consistent with the computing device 400 and/or implemented in a computing device consistent with computing device 400 (or a part thereof, such as, for example, memory 404, etc.). However, the system 100 should not be considered to be limited to the computing device 400, as described below, as different computing devices and/or arrangements of computing devices may be used. In addition, different components and/or arrangements of components may be used in other computing devices.

As shown in FIG. 4, the example computing device 400 includes a processor 402 and a memory 404 coupled to (and in communication with) the processor 402. The processor 402 may include one or more processing units (e.g., in a multi-core configuration, etc.). For example, the processor 402 may include, without limitation, a central processing unit (CPU), a microcontroller, a reduced instruction set computer (RISC) processor, a graphics processing unit (GPU), an application specific integrated circuit (ASIC), a programmable logic device (PLD), a gate array, and/or any other circuit or processor capable of the functions described herein.

The memory 404, as described herein, is one or more devices that permit data, instructions, etc., to be stored therein and retrieved therefrom. In connection therewith, the memory 404 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable media for storing such data, instructions, etc. In particular herein, the memory 404 is configured to store data including, without limitation, genome sequences, patterns, and/or other types of data (and/or data structures) suitable for use as described herein. Furthermore, in various embodiments, computer-executable instructions may be stored in the memory 404 for execution by the processor 402 to cause the processor 402 to perform one or more of the operations described herein (e.g., one or more of the operations of method 500, etc.) in connection with the various different parts of the system 100, such that the memory 404 is a physical, tangible, and non-transitory computer readable storage media. Such instructions often improve the efficiencies and/or performance of the processor 402 that is performing one or more of the various operations herein, whereby such performance may transform the computing device 400 into a special-purpose computing device. It should be appreciated that the memory 404 may include a variety of different memories, each implemented in connection with one or more of the functions or processes described herein.

In the example embodiment, the computing device 400 also includes a presentation unit 406 that is coupled to (and is in communication with) the processor 402 (however, it should be appreciated that the computing device 400 could include output devices other than the presentation unit 406, etc.). The presentation unit 406 may output information (e.g., illustrations of detected edits, etc.), visually or otherwise, to a user of the computing device 400, such as a breeder or other person associated with selection of a nature of edits, etc. It should be further appreciated that various interfaces (e.g., as defined by network-based applications, websites, etc.) may be displayed at computing device 400, and in particular at presentation unit 406, to display certain information to the user. The presentation unit 406 may include, without limitation, a liquid crystal display (LCD), a light-emitting diode (LED) display, an organic LED (OLED) display, an “electronic ink” display, speakers, etc. In some embodiments, presentation unit 406 may include multiple devices. Additionally or alternatively, the presentation unit 406 may include printing capability, enabling the computing device 400 to print text, images, and the like on paper and/or other similar media.

In addition, the computing device 400 includes an input device 408 that receives inputs from the user (i.e., user inputs) such as, for example, selections of edits and genomes to which the edits are directed, etc. The input device 408 may include a single input device or multiple input devices. The input device 408 is coupled to (and is in communication with) the processor 402 and may include, for example, one or more of a keyboard, a pointing device, a mouse, a stylus, a touch sensitive panel (e.g., a touch pad or a touch screen, etc.), or other suitable user input devices. In addition, the input device 408 may include, without limitation, sensors disposed and/or associated with the genome editor 102 and/or the sequencer 104. It should be appreciated that in at least one embodiment an input device 408 may be integrated and/or included with a presentation unit 406 (e.g., a touchscreen display, etc.).

Further, the illustrated computing device 400 also includes a network interface 410 coupled to (and in communication with) the processor 402 and the memory 404. The network interface 410 may include, without limitation, a wired network adapter, a wireless network adapter, a mobile network adapter, or other device capable of communicating to one or more different networks (e.g., one or more of a local area network (LAN), a wide area network (WAN) (e.g., the Internet, etc.), a mobile network, a virtual network, and/or another suitable public and/or private network capable of supporting wired and/or wireless communication among two or more of the parts illustrated in FIG. 1, etc.), including with other computing devices used as described herein.

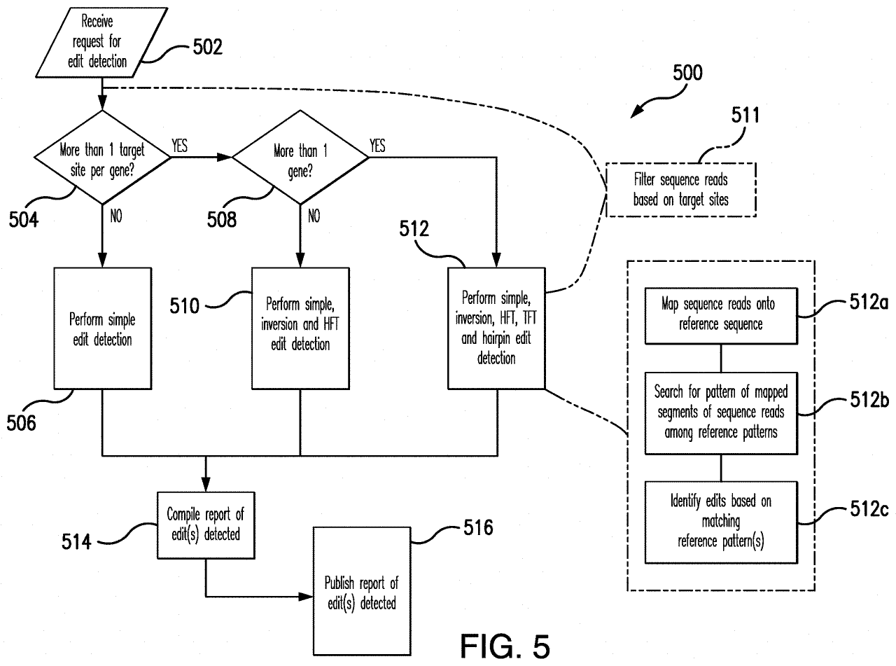

FIG. 5 illustrates an example method 500 for use in detecting one or more edits in output genomes (based on sequence reads associated therewith) relative to reference sequences. The example method 500 is described herein in connection with the computing device 106 of the system 100, and is also described with reference to computing device 200. However, it should be appreciated that the methods herein are not limited to the system 100, or computing device 200. And, likewise, the systems and computing devices described herein are not limited to the example method 500.

Generally, and prior to the method 500, a user has defined one or more edits for an input genome (of a selected organism), and provided the input genome and the one or more defined edits to the genome editor 102, resulting in an edited output genome. In turn, in connection with the sequencer 104, a reference sequence for the genome may be generated and stored in the data structure 108, identifying potential target positions for edits in the genome (e.g., gRNA target positions, etc.) and sequence reads for the genome. The data structure 104 may also include reference sequences for other genome obtained in a similar manner, or otherwise (e.g., as described above in connection with the system 100, etc.).

Then in the method 500, the computing device 106 receives a request to detect edits for a genome, at 502 (e.g., an edited, output genome from the editor 102 and sequencer 104, etc.). The request may include the reference sequence associated with the genome and/or corresponding sequence reads as well as characteristics of edits associated with the reference sequence. Or, the request may include identifiers of the reference sequence and/or sequence reads therefore in the data structure 108, whereby the computing device 106 is permitted to retrieve the reference sequence and/or sequence reads. The request may further include certain data related to the editing of the output genome, including, for example (and without limitation), a number of target sites associated with the editing of the output genome (e.g., a number of gRNA or other editing technologies employed to perform the editing, etc.), sample descriptions of edits, etc. In turn, the computing device 106 determines, at 504, whether there was more than one target site per gene/locus (e.g., whether there was more than one gRNA targeting each gene/locus, etc.) employed in the genome editing (e.g., at genome editor 102, etc.), or not (e.g., based on application of an initial logic, etc.) (broadly, if there was more than one target site for edit per gene).

If there was not more than one target site per gene/locus employed in the genome editing, the computing device 106 performs detection for simple edits in the output genome, at 506. In particular, the computing device 106 maps the sequence reads of the output genome, for example, as read by the sequencer 104 (e.g., via SASi, etc.), onto the reference sequence. The simple edits will include, for example, small insertions or deletions, etc. Consequently, the computing device 106 identifies an additional sequence as indicative of an insertion, and a missing sequence as indicative of a deletion, etc.

Alternatively, if the computing device 106 determines, at 504, that there was more than one target site per gene/locus employed in the genome editing (e.g., at genome editor 102, etc.) (e.g., more than one gRNA targeting each gene/locus, etc.), the computing device 106 determines, at 508, whether there was more than one gene/locus employed in the genome editing (e.g., at genome editor 102, etc.). If there was not, the computing device 106 performs detection for simple, inversion, and HFT edits in the output genome, at 510. However, if there is more than one gene/locus, at 508, the computing device 106 performs detection for simple, inversion, HFT, TFT, and hairpin edits, at 512, in the output genome.

For illustration, potential steps included in step 512 are shown in FIG. 5 in the box denoted by dotted lines. In this example embodiment, the computing device 106 maps, at 512a, for example, the sequence reads, from the sequencer 104 (or ones thereof), to the reference sequence (e.g., via an alignment program or technique, etc.). In particular, when a sequence read does not map onto a target site, or a site directly neighboring a target site, the sequence read may be discarded from the method 500 (yet counted for reasons explained below). Conversely, when the sequence read maps to a target site, the sequence read is retained. Thereafter, the computing device 106 maps the retained local assignment of the sequence reads onto the reference sequence, which forms patterns by locations and/or orientations of the segments of the sequence reads, etc. For instance, the sequence reads may be characterized as having two segments mapped onto two spaced apart locations of the reference sequence, with one segment in an opposite orientation.

Once the pattern or read type of the sequence reads of the output genome are defined, the computing device 106 searches, at 512b, for the pattern(s) among the reference patterns in the data structure 108, in whole or in part.

In this example embodiment, when the edit pattern(s) of the sequence reads of the output genome matches one or more reference patterns (or reference edit patterns) in the data structure 108 (e.g., one of the reference read types, etc.), the computing device 106 identifies, at 512c, the edit(s) based on the matching reference pattern(s). It should be appreciated that one reference pattern may be identified, or multiple reference patterns may be identified (e.g., indicating a composite, etc.) (depending on the types of read types included in the data structure 108), etc. It should further be appreciated that when the pattern(s) produced from mapping the sequence reads to the reference sequence does not match a reference pattern in the data structure 108, the computing device 106 may determine the edit pattern is not included in the data structure 108 and add the pattern to the data structure 108, and/or request user review of the pattern and/or edit in connection with adding the pattern to the data structure, as a new reference read type, etc.

In connection therewith, in some implementations of the method 500, the computing device 106 may optionally (as indicated by the broken lines in FIG. 5) filter the sequence reads, at 511, prior to performing the detection at 512 (or potentially prior to step 504, or otherwise as suitable). In doing so, the filtering may be based on the target sites as defined, for example, by the gRNAs, etc., whereby sequence reads overlapping the target site are retained and other sequence reads are discarded. In doing so, the detection, as described below, is directed to the target sites.

While the steps 511 and 512a-c of the method 500 are described with reference to the detection of inverse, HFT, TFT, and hairpin edits, at 512, the steps 511 and 512a-c should be understood to also be performed, for example, as part of steps 506 and 510 in any suitable order to perform the detections thereof.

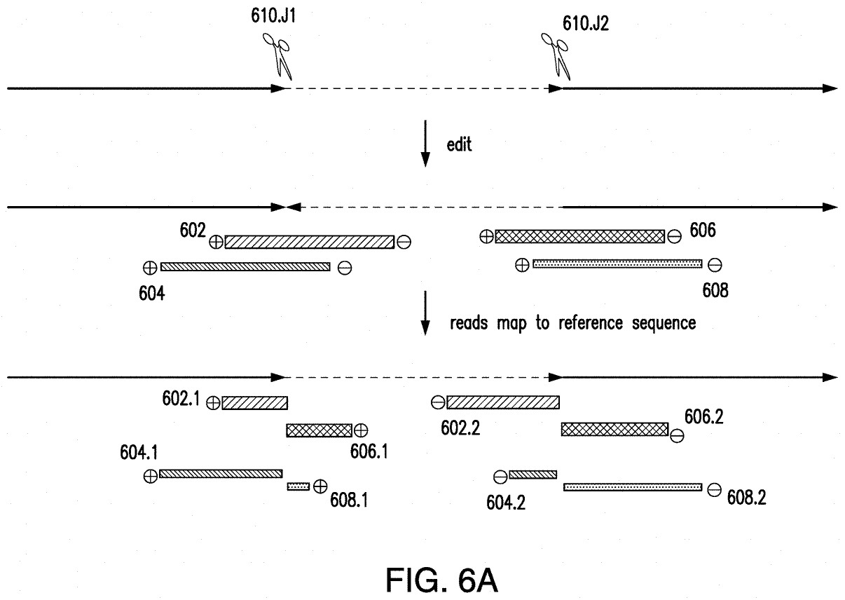

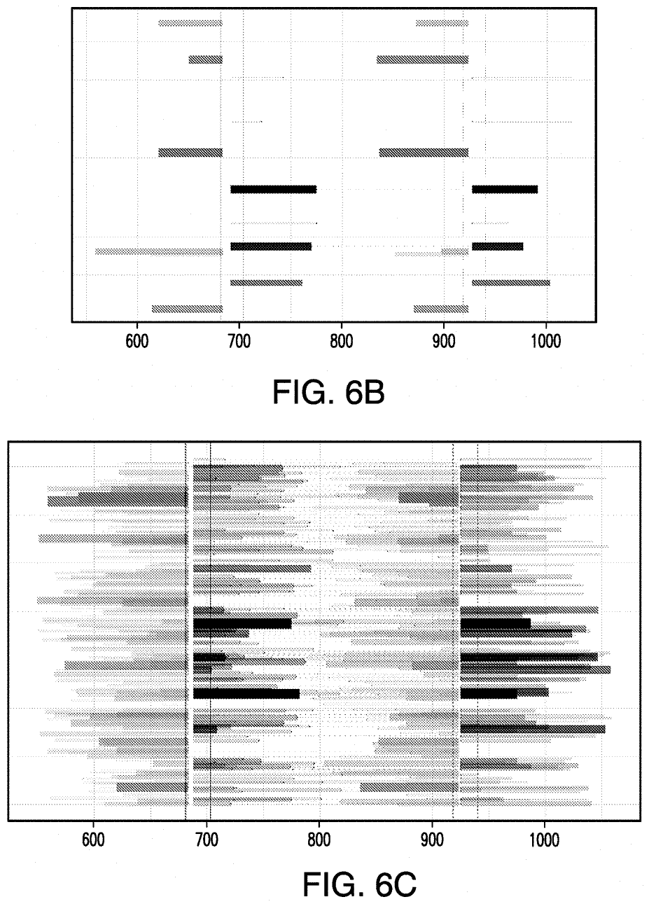

FIG. 6A schematically illustrates an example detection that may be performed in the method 500, at step 510, by the computing device 106, of an inversion edit to an input sequence of an input genome (e.g., as input to the genome editor 102, etc.). Specifically, as shown, the input sequence of the input genome is edited at junctions 610.J1 and 610.J2 and sequenced, for instance, by the sequencer 104, to provide multiple sequence reads. And then, the sequence reads are mapped back to a reference sequence for the input genome. In this example, for purposes of illustration, four sequence reads 602, 604, 606, and 608 are provided, including two sequence reads (602 and 604) overlapping junction 610.J1 and two sequence reads (606 and 608) overlapping junction 610.J2, between which an inversion edit is made. Further, reference indicators, in the form of either circled plus signs or circled minus signs, are included in FIG. 6A at each of the sequence reads 602-608 to denote the orientation of segments 602.1 and 602.2; 604.1 and 604.2; 606.1 and 606.2; and 608.1 and 608.2 corresponding to the reference sequence, as part of the alignment of the sequence reads 602-608 to the reference sequence.

The sequence reads 602-608 are split, in this example, at the junctions 610.J1 and 610.J2, as apparent from the segments 602.1 and 602.2; 604.1 and 604.2; 606.1 and 606.2; and 608.1 and 608.2. In connection therewith, the orientation of segments 602.2, 604.2, 606.1 and 608.1 are all reversed when mapped to the reference sequence, relative to the orientation of the segments of the sequence reads. As such, the computing device 106 is configured, based on the locations and orientations of the segments 602.1-608.2 of the sequence reads 602-608, to detect a completed inversion edit between the junctions 610.J1 and 610.J2.

In this example (FIG. 6A), only four sequence reads 602-608 and two junctions 610.J1 and 610.J2, and a mapping thereof, are illustrated. FIGS. 6B-C illustrate charts having a substantially increased number of sequence reads, based on edits to an input genome, for example, from a LASi sequencing technique (e.g., by the sequencer 104, etc.). These charts illustrate observed data, as compared to the illustrative diagram of FIG. 6A (based on the same edit discussed above with regard to FIG. 6A and the reference sequence therein). In connection therewith, the charts of FIGS. 6B-C each illustrate a pattern (as described above) identified (or recognized or recognizable) by the computing device 106, relative to the two junctions 610.J1 and 610.J2 of the edited input sequence. It should be appreciated that the locations or coordinates of the junctions are shown on the bottom axis of each of the charts, which are indicated as base pairs (or nucleotides). As such, for example, in FIG. 6C, the junction 610.J1 is at (about) 685 nucleotides, whereby not only the particular edit is detected, by the computing device 106, but also the specific location of the edit, as described more below.

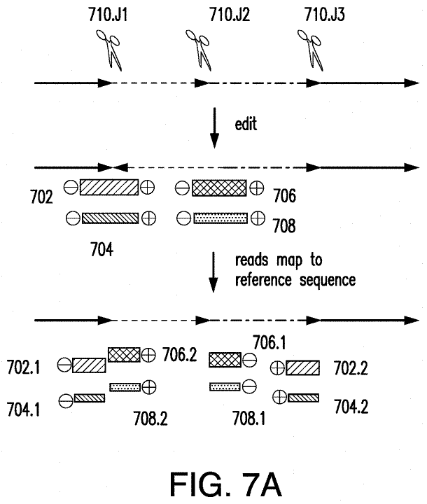

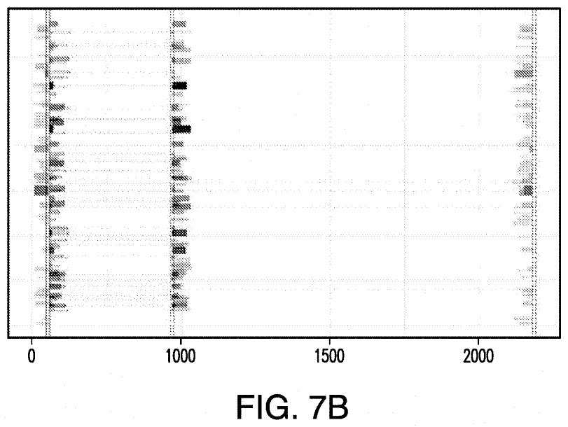

FIG. 7A schematically illustrates another example detection that may be performed in the method 500, at step 510, by the computing device 106, of a homolog fragment targeting (HFT) edit to an input sequence of an input genome. Specifically, as shown, the input sequence is edited at junctions 710.J1, 710.J2 and 710.J3 and sequenced, by the sequencer 104, and described above, to provide four sequence reads 702, 704, 706, and 708. And then, the sequence reads 702-708 are mapped back to a reference sequence for the input genome. As shown, the output genome, from the sequencer 104, includes, in this example (for purposes of illustration), the four sequence reads 702-708. Two of the sequence reads 702 and 704 overlap the junction 710.J1, and two of the sequence reads 706 and 708 overlap the junction 710.J2.

In particular, the sequence reads 702-708 are split, in this example, at the junctions 710.J1 and 710.J2, as indicated from the segments 702.1 and 702.2; 704.1 and 704.2; 706.1 and 706.2; and 708.1 and 708.2. Reference indicators, again in the form of either circled plus signs or circled minus signs, are included to denote the orientation of the segments of the sequence reads (and as mapped onto the reference sequence). In connection therewith, the orientations of segments 702.2, 704.2, 706.1 and 708.1 are all reversed when mapped to the reference sequence, while the orientations of segments 702.1, 704.1, 706.2, and 708.2 are not reversed. As a result, the computing device 106 is configured, based on the locations and orientations of the segments 702.1-708.2 of the sequence reads 702-708, to detect a completed HFT edit, wherein a part of the input sequence between junctions 710.J1 and 710.J2 is swapped with a part of the input sequence between junctions 710.J2 and 710.J3, with the part of the input sequence between junctions 710.J2 and 710.J3 being inversed.

In this example (FIG. 7A), only four sequence reads 702-708 and three junctions 710.J1-710.J3, and a mapping thereof, are illustrated for ease of reference. FIG. 7B illustrates a chart having a substantially increased number of sequence reads, based on edits to an input genome, for example, from a LASi sequencing technique (e.g., by the sequencer 104, etc.). The chart illustrates observed data, as compared to the illustrative diagram of FIG. 7A (based on the same edit discussed above with regard to FIG. 7A and the reference sequence therein). In connection therewith, the chart of FIG. 7B illustrates a pattern (as described above) identified by the computing device 106, relative to the three junctions 710.J1, 710.J2 and 710.J3 of the input sequence.

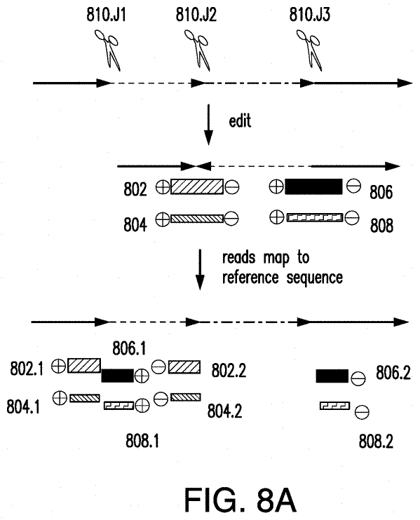

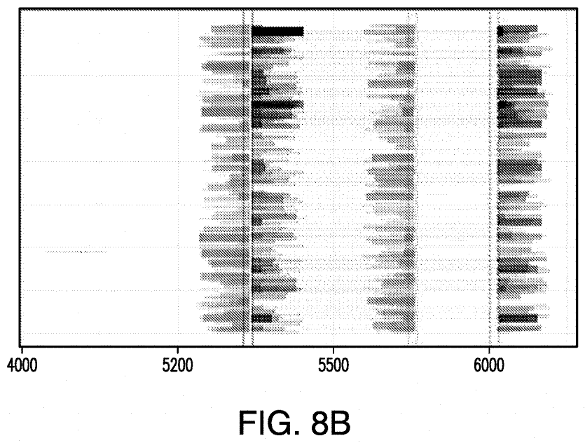

FIG. 8A schematically illustrates still another example detection that may be performed in the method 500 (at step 510), by the computing device 106, of an inversion edit and a deletion edit to an input sequence of an input genome. Specifically, as shown, the input sequence is edited at junctions 810.J1, 810.J2, and 810.J3. As shown, then, sequencing of the output genome by the sequencer 104 results in four sequence reads 802, 804, 806, and 808. In connection therewith, two of the sequence reads 802 and 804 overlap junction 810.J2, and two of the sequence reads 806 and 808 overlap junction 810.J3.

In particular, in this example, the sequence reads 802-808 are split at the junctions 810.J2 and 810.J3, as indicated from the segments 802.1 and 802.2; 804.1 and 804.2; 806.1 and 806.2; and 808.1 and 808.2. Reference indicators, in the form of either circled plus signs or circled minus signs are again included at each of the sequence reads 802-808 to denote the relative orientation of the segments 802.1-808.2 of the sequence reads (and the mapping onto the reference sequence). In connection therewith, the orientations of segments 802.2, 804.2, 806.1, and 808.1 are all reversed when mapped to the reference sequence, while the orientations of segments 802.1, 804.1, 806.2 and 808.2 are not reversed. As a result, the computing device 106 understands that the sequence reads are mapped onto the upstream of the junctions 810.J1 and 810.J2, and are mapped onto downstream of the junctions 810.J1 and 810.J3, indicating that the sequence reads between the junctions 810.J2 and 810J3 in the reference sequence is deleted in editing. In connection therewith, the computing device 106 is configured, based on the locations and orientations of the segments 802.1-808.2 of the sequence reads 802-808, and the lack of sequence reads between junctions 810.J2 and 810.J3, to detect completed inversion and deletion edits, wherein a part of the reference sequence between junctions 810.J1 and 810.J2 is inverted and a part of the reference sequence between junctions 810.J2 and 810.J3 is deleted.

In this example (FIG. 8A), only four sequence reads 802-808 and three junctions 810.J1-810.J3, and a mapping thereof, are illustrated for ease of reference. FIG. 8B illustrates a chart having a substantially increased number of sequence reads, based on edits to an input genome, for example, from a LASi sequencing technique (e.g., by the sequencer 104, etc.). The chart illustrates observed data, as compared to the illustrative diagram of FIG. 8A (based on the same edits discussed above with regard to FIG. 8A and the reference sequence therein). In connection therewith, the chart of FIG. 8B illustrates a pattern (as described above) identified by the computing device 106, relative to the three junctions 810.J1, 810.J2 and 810.J3 of the input sequence.

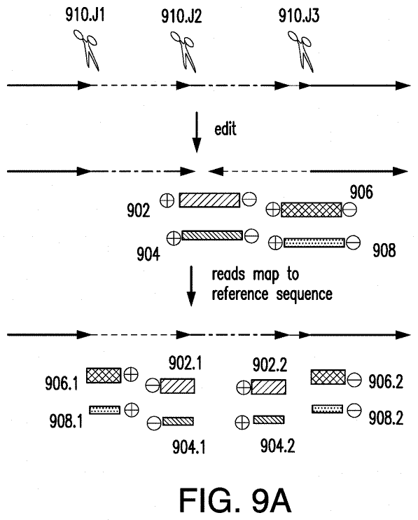

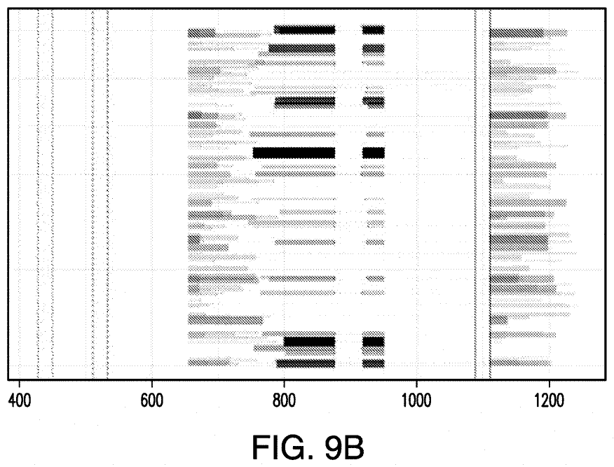

FIG. 9A schematically illustrates a further example detection that may be performed in the method 500, at step 510, by the computing device 106, of a HFT edit and a deletion edit to an input sequence of an input genome. Specifically, as shown, the input sequence is edited at junctions 910.J1, 910.J2, and 910.J3. Then, sequencing of the output genome by the sequencer 104 results in four sequence reads 902, 904, 906, and 908. Two of the sequence reads 902 and 904 overlap junction 910.J2, and two of the sequence reads 906 and 908 overlap junction 910.J3.

In particular, the sequence reads 902-908 are split, in this example, at the junctions 910.J2 and 910.J3, as indicated from the segments 902.1 and 902.2; 904.1 and 904.2; 906.1 and 906.2; and 908.1 and 908.2. Again, reference indicators, in the form of either circled plus signs or circled minus signs, are included to denote the relative orientation of the segments 902.1-908.2 of the sequence reads 902-908. In connection therewith, the orientations of segments 902.1, 904.1, 906.1 and 908.1 are all reversed when mapped to the reference sequence, and the orientations of segments 902.2, 904.2, 906.2 and 908.2 are not reversed. And, the computing device 106 recognizes that a part of the input sequence between the junctions 910.J1 and 910.J2 is swapped with a part between the junctions 910.J2 and 910.J3. What's more, as shown, the edit includes a deletion of part of the input sequence extending between the junctions 910.J2 and 910.J3. As a result, the computing device 106 is configured, based on the locations and orientations of the segments 902.1-908.2 of the sequence reads 902-908, and the swap, to detect a completed HFT edit and deletion edit wherein a part of the input sequence between junctions 910.J1 and 910.J2 is inverted and transposed with the part of the input sequence between junctions 910.J2 and 910.J3, less a deletion of a sub-part (as shown in FIG. 9A) of that part of the input sequence between junctions 910.J2 and 910.J3.

In this example (FIG. 9A), again, only four sequence reads 902-908 and three junctions 910.J1-910.J3, and a mapping thereof, are illustrated (for ease of reference). FIG. 9B illustrates a chart having a substantially increased number of sequence reads, based on edits to an input genome, for example, from a LASi sequencing technique (e.g., by the sequencer 104, etc.). The chart illustrates observed data, as compared to the illustrative diagram of FIG. 9A (based on the same edits discussed above with regard to FIG. 9A and the reference sequence therein). In connection therewith, the chart of FIG. 9B illustrates a pattern (as described above) identified by the computing device 106, relative to the three junctions 910.J1, 910.J2 and 910.J3 of the input sequence.

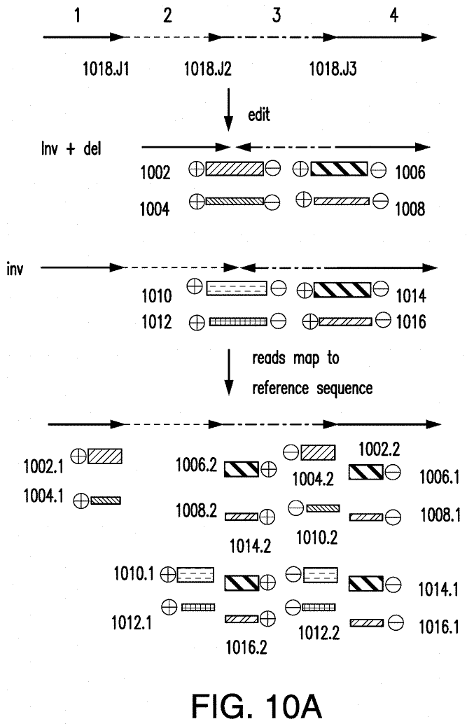

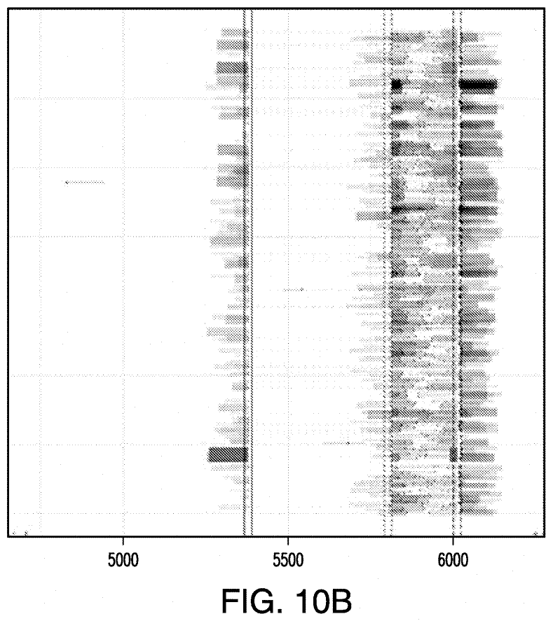

FIG. 10A schematically illustrates yet another example detection that may be performed in the method 500, at step 510, by the computing device 106, of inversion and deletion edits to an input sequence of an input genome, and also an inversion edit to the input sequence. In particular, when edit machinery (e.g., gRNA, etc.) is introduced to more than one cell, or targeting more than one gene for edit, the edit is not consistent among the different cells or genes. In such cases, as shown, multiple instances of the input sequence are edited. When the edit is attempted or performed at junctions 1018.J1, 1018.J2 and 1018.J3, by the associated edit machinery, it is possible for deletion of the part of the input sequence between junctions 1018.J1 and 1018.J2, and inversion of the part of the input sequence between junctions 1018.J2 and 1018.J3, as illustrated in FIG. 10A by “Inv+del.” Or, alternatively, it is possible for the cut site at junction 1018.J1 to be ineffective or to heal or to otherwise not result in an edit to the adjacent part of the input sequence between junction 1018.J1 and 1018.J2, while the inversion of the part of the input sequence between junctions 1018.J2 and 1018.J3 is completed, as illustrated in FIG. 10A by “inv.” As such, the computing device 106 detects multiple, inconsistent edits. And, sequence reads for both sets of edits are mapped back to a reference sequence for the input genome to provide two different patterns. In connection therewith, the sequencer 104 provides eight sequence reads 1002, 1004, 1006, 1008, 1010, 1012, 1014, and 1016. Four of the sequence reads 1002, 1004, 1010, and 1012 overlap the junction 1018.J2, while four of the sequence reads 1006, 1008, 1014, and 1016 overlap the junction 1018.J3.

In particular, the sequence reads 1002-1016 are split at the junctions 1018.J2 and 1018.J3, as indicated from resulting segments 1002.1 and 1002.2; 1004.1 and 1004.2; 1006.1 and 1006.2; 1008.1 and 1008.2; 1010.1 and 1010.2; 1012.1 and 1012.2; 1014.1 and 1014.2; and 1016.1 and 1016.2. Again, reference indicators, in the form of either circled plus signs or circled minus signs are included to denote the orientation of the segments 1002.1-1016.2 of the sequence reads 1002-1016. In connection therewith, the computing device 106 detects a pattern associated with an inversion edit, as illustrated by the sequence reads 1010, 1012, 1014, and 1016, and also a pattern associated with inversion plus deletion edits, as illustrated by the sequence reads 1002, 1004, 1006, and 1008. As a result, the computing device 106 is configured, based on the locations and orientations of the segments 1002.1-1016.2, to detect two different sets of edits performed, one as an inversion plus deletion edit, and the other as an inversion edit.

FIG. 10B illustrates a chart having a substantially increased number of sequence reads, based on edits to an input genome, for example, from a LASi sequencing technique (e.g., by the sequencer 104, etc.). The chart illustrates observed data, as compared to the illustrative diagram of FIG. 10A (based on the same edits discussed above with regard to FIG. 10A and the reference sequence therein). In connection therewith, the chart of FIG. 10B illustrates particular patterns (as described above) identified by the computing device 106, relative to the three junctions 1018.J1, 1018.J2 and 1018.J3 of the input sequence.

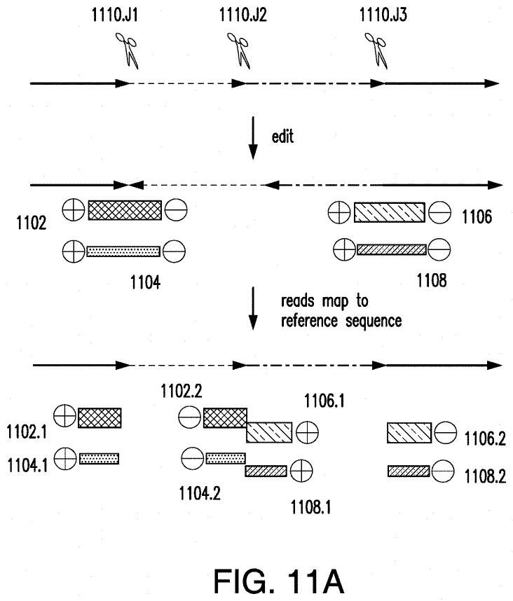

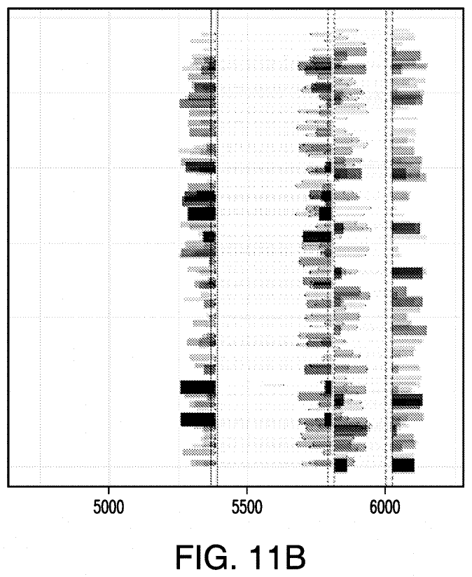

FIG. 11A schematically illustrates another example detection that may be performed in the method 500, at step 510, by the computing device 106, of multiple inversion edits to an input sequence of an input genome. Specifically, as shown, the input sequence is edited at junctions 1110.J1, 1110.J2, and 1110.J3. As shown, then, sequencing of the output genome by the sequencer 104 results in four sequence reads 1102, 1104, 1106, and 1108. Two sequence reads 1102 and 1104 overlap junction 1110.J1, and two sequence reads 1106 and 1108 overlap junction 1110.J3.

In particular, the sequence reads 1102-1108 are split at the junctions 1110.J1 and 1110.J3, as indicated from segments 1102.1 and 1102.2; 1104.1 and 1104.2; 1106.1 and 1106.2; and 1108.1 and 1108.2. Reference indicators, again, in the form of either circled plus signs or circled minus signs are included to denote the orientation of the segments 1102.1-1108.2 of the sequence reads 1102-1108. In connection therewith, the orientations of segments 1102.2, 1104.2, 1106.1 and 1108.1 are all reversed when mapped to the reference sequence, and the orientations of segments 1102.1, 1104.1, 1106.2 and 1108.2 are not reversed. And, the computing device 106 recognizes that a part of the input sequence between the junctions 1110.J1 and 1110.J2 and a part of the input sequence between the junctions 1110.J2 and 1110.J3 are both inverted. As a result, the computing device 106 is configured, based on the locations and orientations of the segments 1102.1-1108.2 of the sequence reads 1102-1108, to detect inversion edits of the two parts between the junctions 1110.J1-1110.J3.

In this example (FIG. 11A), again, only four sequence reads 1102-1108 and three junctions 1110.J1-1110.J3 of the input sequence, and a mapping thereof, are illustrated. FIG. 11B illustrates a chart having a substantially increased number of sequence reads, based on edits to an input genome, for example, from a LASi sequencing technique (e.g., by the sequencer 104, etc.). The chart illustrates observed data, as compared to the illustrative diagram of FIG. 11A (based on the same edit discussed above with regard to FIG. 11A and the reference sequence therein). In connection therewith, the chart of FIG. 11B illustrates a pattern (as described above) identified by the computing device 106, relative to the three junctions 1110.J1, 1110.J2 and 1110.J3 of the input sequence.

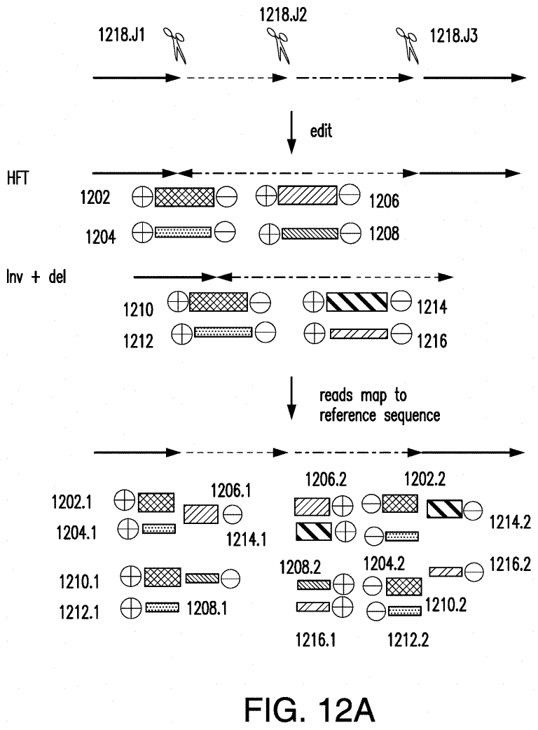

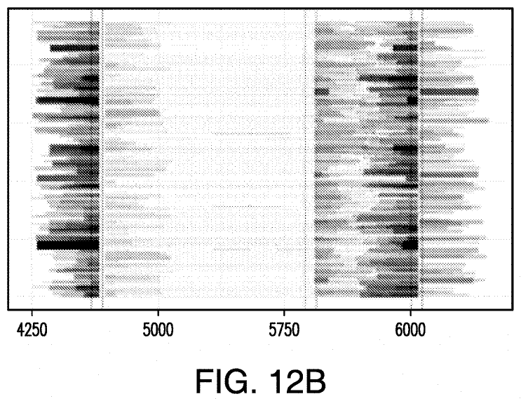

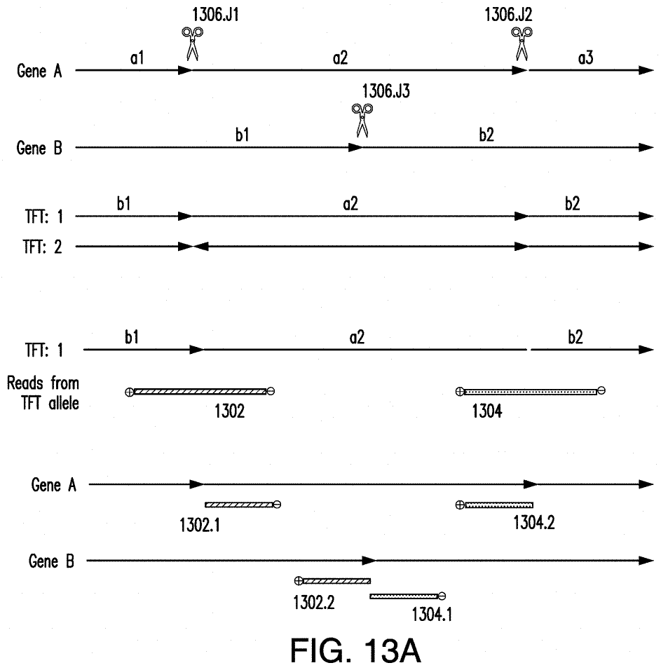

FIG. 12A schematically illustrates a still further example detection that may be performed in the method 500, at step 510, by the computing device 106, of HFT, inversion, and deletion edits to an input sequence of an input genome. Specifically, as shown, the input sequence is edited and sequenced, resulting in eight sequence reads 1202, 1204, 1206, 1208, 1210, 1212, 1214, and 1216. Four of the sequence reads 1202, 1204, 1210, and 1212 overlap the junction 1218.J1, two of the sequence reads 1206 and 120 overlap the junction 1218.J2, and two of the sequence reads 1214 and 1216 overlap junction 1218.J3. Like in FIG. 10A, there are multiple genes (or alleles) shown in FIG. 12A, thereby indicating (from the associated patterns) that the input gene was edited in at least two different manners.

In particular, the sequence reads 1202-1216 are split at the junctions 1218.J1-1218.J3, as indicated from the segments 1202.1 and 1202.2; 1204.1 and 1204.2; 1206.1 and 1206.2; 1208.1 and 1208.2; 1210.1 and 1210.2; 1212.1 and 1212.2; 1214.1 and 1214.2; and 1216.1 and 1216.2. Reference indicators, in the form of either circled plus signs or circled minus signs, are included to again denote the orientation of the segments 1202.1-1216.2 of the sequence reads 1202-1216. In connection therewith, the computing device 106 detects a pattern associated with both an HFT edit, as illustrated by the sequence reads 1202-1208, and also a pattern associated with inversion plus deletion edits, as illustrated by the sequence reads 1210-1216. As a result, the computing device 106 is configured, based on the locations and orientations of the segments 1202.1-1216.2, to detect two different sets of edits performed, one as inversion plus deletion edits, and the other as an inversion edit.

FIG. 12B illustrates a chart having a substantially increased number of sequence reads, based on edits to an input genome, for example, from a LASi sequencing technique (e.g., by the sequencer 104, etc.). The chart illustrates observed data, as compared to the illustrative diagram of FIG. 12A (based on the same edit discussed above with regard to FIG. 12A and the reference sequence therein). In connection therewith, the chart of FIG. 12B illustrates patterns (as described above) identified by the computing device 106, relative to the three junctions 1218.J1, 1218.J2, and 1218.J3 of the input sequence.

It should be appreciated that the above detection examples are provided for illustration with regard to the method 500, and are not limiting of the detection performed by the computing device 106 for simple, inversion, and/or HTF edits, as other combinations of the above edits may be detectable by the computing device 106 consistent with the pattern recognition described above.

Referring again to FIG. 5 and method 500, if the computing device 106 determines, at 508, that more than one gene is included in the genome editing, the computing device 106 performs detection, at 512, for simple, inversion, HFT, TFT, and hairpin edits in the output genome. In particular, the computing device 106 maps the sequence reads for the genes, as read by the sequencer 104 (e.g., via LASi, etc.), onto the reference sequence for the input genome.

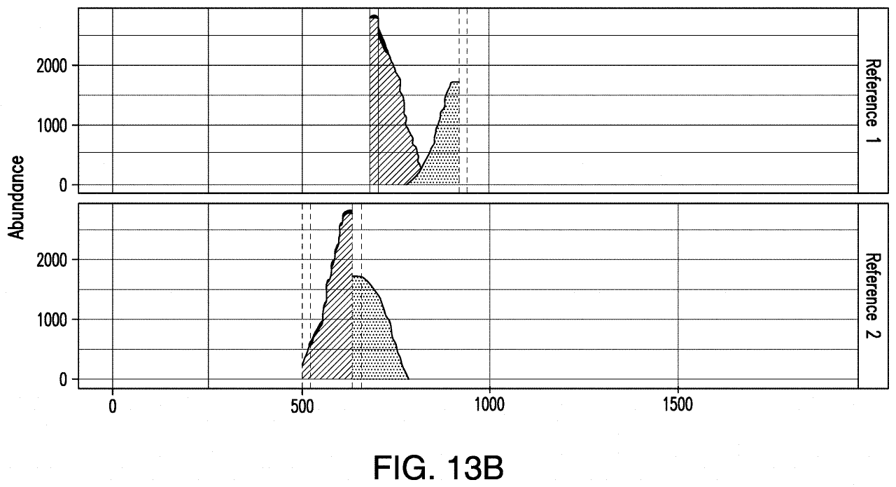

FIGS. 13A-B, then, illustrate an example detection, by the computing device 106, of TFT edits to two input sequences (Gene A and Gene B). Specifically, as shown, the input sequences are exposed to editing to provide the TFT:1 and TFT:2, as read by the sequencer 104. Sequence reads therefore are mapped back to reference sequences for the genes. As shown, as part of the editing (e.g., via gRNAs, etc.), there are three target sites: two target sites (or junctions 1306.J1 and 1306.J2) in the Gene A and one target site (or junction 1306.J3) in Gene B. After the edit, the genes are subjected to reads from the sequencer 104, providing two sequence reads, 1302 and 304, for the two junctions of translocation edits, where a part of gene/locus A between two gRNAs targeting the gene/locus is cut and is inserted into the cut site in gene/locus B in the original orientation.

When the sequence reads 1302 and 1304 are read back onto the input sequence for Gene A and the input sequence for Gene B, the mapping is illustrated in FIG. 13A, with segments 1302.1 and 1304.2 mapping onto the input sequence of Gene A and segments 1302.2 and 1304.1 mapping onto the input sequence of Gene B. Reference indicators, in the form of either circled plus signs or circled minus signs, are included to again denote the orientation of the segments 1302.1-1304.2 of the sequence reads 1302 and 1304. As such, it is apparent that the orientations of the sequence reads 1302 and 1304 (and their segments 1302.1-1304.2) are consistent with the input sequence, whereby there is no inversion edits of the genes. In connection therewith, the computing device 106 identifies a pattern and searches for that pattern in the data structure 108. As a result, the computing device 106 is configured, based on the locations and orientations of the sequence reads 1302 and 1304 and the segments 1302.1-1304.2 thereof, relative to the junctions 1306.J1, 1306.J2, and 1306.J3, to detect a pattern between Gene A and Gene B indicating a TFT edit.

FIG. 13B illustrates a chart having a substantially increased number of sequence reads, based on the genes of FIG. 13A. In connection therewith, the chart of FIG. 13B illustrates a pattern (as described above) identified by the computing device 106, relative to the three junctions 1306.J1, 1306.J2, and 1306.J3 of the genes.

Optionally in the method 500, the computing device 106, after detecting the edit(s) between the input genome and the output genome, may determine if the output genome is associated with a stable organism and makes a zygosity call.

With reference again to FIG. 5, after the computing device 106 performs the appropriate detection, as described above (and in view of the corresponding logic), by identifying one or more editing patterns apparent from the output genome, with each edit defined at the coordinates of the reference sequence (e.g., based on the mapping, etc.), the computing device 106 compiles, at 514, a report of the detected edits. The report may include, for example, a specific location of the edits and description of the type of edits (e.g., a deletion edit, an inversion edit, a HFT edit, a TFT edit, combinations thereof, etc.), etc. The computing device 106 then publishes, at 516, the report to a user, another computing device, etc. That said, the publication may include displaying the report to the user, or transmitting the report (e.g., via email, etc.) (e.g., in response to an API call, etc.) to the user associated with the request received at 502.

An example output from the computing device 106 is shown in Table 2. As shown, Table 2 includes four reports, which represent four different edit detections via the method 500. The sample identifier (ID) includes an identification of the particular sample for which detection was requested. The Reads column includes locus identifiers for the edits detected in the output genome, and the type includes a description or identification of the type of edit detected. The call column (“Call”) includes coordinates of a region or part of the sequence involved in the edit. The coordinate column (“Coor”) includes the coordinates to rebuild the edited sequences from the reference sequence. Also, the reference reads column (“Ref. Reads”) includes a number of sequence reads, and the Edit Read % column indicates a percentage of the “reads” associated with the edit to all reads for the sequence (e.g., reads/(reads+reference reads)×100, etc.). Generally, the Edit Read % is an indication of the heredity of the detected edits into later generations of the plant/organism.

TABLE 2

| | | | | | | Edit |
|---|---|---|---|---|---|---|
| Sample | | | | | Ref. | Read |
| ID | Reads | Type | Call | Coor | Reads | (%) |
| S021 | 18365 | Inversion | 6016 . . . 5388 | 1 . . . 5389, | 12197 | 60.1 |
| | | | | 6016 . . . 5388, | | |
| | | | | 6020 . . . 7812 | | |
| S058 | 6903 | Inversion + | 6016 . . . 5814 | 1 . . . 5384, | 5888 | 54 |
| | | Deletion | | 6016 . . . 5814, | | |
| | | | | 6024 . . . 7812 | | |
| S123 | 32460 | (Inversion + | (6013 . . . 5812); | (1 . . . 5379, | 119 | 99.6 |
| | | Deletion); | (6013 . . . 5812) | 6013 . . . 5812, | | |
| | | (Inversion) | | 6025 . . . 7812); | | |
| | | | | (1 . . . 5808, | | |
| | | | | 6013 . . . 5812, | | |
| | | | | 6025 . . . 7812) | | |
| S138 | 29404 | (HFT); | (Inversion between | (1 . . . 5384, | 28353 | 50.9 |
| | | (Inversion + | 6015 . . . 5814 | 6015 . . . 5814, | | |
| | | Deletion) | inserted into | 5390 . . . 5813, | | |
| | | | 5384 . . . 5390); | 6016 . . . 7812); | | |
| | | | (5805 . . . 5387) | (1 . . . 5382, | | |
| | | | | 5805 . . . 5387, | | |
| | | | | 6020 . . . 7812) | | |

It should be appreciated that the format and/or content of the output may be different in other method embodiments, as defined by a user or otherwise, etc.

In view of the above, the systems and methods herein provide advanced edit detection techniques based on sequence reads and associated patterns. In particular, by relying on the target site of any genome editing technology or mechanism, the detection engine herein is able to identify the underlying target sites, within the broader output genomes, at which edits exist, if successful. The detection engine then relies on sequence reads, from a sequencer, at or around the target site or sites, for the output genomes, and maps the sequence reads back onto reference sequences for the genomes, etc. In doing so, the detection engine forms patterns defined by the segments of the sequence reads, specifically, the locations and orientations. The patterns are then compared and/or matched/searched relative to multiple reference patterns or read types indicating particular edits (or combinations of edits). The patterns may match particular reference patterns, thereby indicating the edits associated with the reference patterns, or to multiple patterns, thereby indicating multiple edits and/or inconsistent edits amongst cells/genes, etc. In this manner, the detection engine provide a flexible way of detecting edits in output genomes, which requires no knowledge of what specific edits are actually being attempted or if the attempted edits were successful (e.g., there is no matching the sequence reads to an “expected” output which is built or defined based on the attempted edits, etc.). It should further be appreciated that the detection engine may, for certain edits, permit the detection while mapping sequence reads at less than each of the target sites of the edit(s), as defined by the gRNA (or other editing mechanisms) (as shown in FIG. 9A, for example).

The functions described herein, in some embodiments, may be described in computer executable instructions stored on a computer readable media, and executable by one or more processors. The computer readable media is a non-transitory computer readable media. By way of example, and not limitation, such computer readable media can include RAM, ROM, EEPROM, CD-ROM or other optical disk storage, magnetic disk storage or other magnetic storage device, or any other medium that can be used to carry or store desired program code in the form of instructions or data structures and that can be accessed by a computer. Combinations of the above should also be included within the scope of computer-readable media

It should also be appreciated that one or more aspects of the present disclosure transform a general-purpose computing device into a special-purpose computing device when configured to perform the functions, methods, and/or processes described herein.

As will be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the following operations: (a) receiving, by a computing device, a request to detect at least one edit in an output genome (or edited genome), the output genome (or edited genome) based on an input genome (e.g., an input wild type genome, etc.) and one or more edits to the input genome, wherein one or more reference sequences are representative of the input genome; (b) detecting, by the computing device, the at least one edit of the one or more edits, based on sequence reads mapped onto the one or more reference sequences relative to one or more reference edit patterns; and (c) reporting, by the computing device, the detected at least one edit.

As will also be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the following operations: (a) receiving a request to detect at least one edit in an edited genome, the edited genome based on an input wild type genome and one or more edits to the input wild type genome, wherein one or more reference sequence(s) is representative of the input wild type genome; (b) detecting the at least one edit of the one or more edits, based on sequence reads mapped to the one or more reference sequence(s), in terms of one or more reference edit patterns; and (c) reporting the detected at least one edit.

As will also be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the following operations: (a) receiving a request to identify at least one edit in an output genome, the output genome based on an input genome and one or more edits to the input genome; (b) mapping multiple sequence reads from the output genome onto one or more reference sequences, wherein the one or more reference sequences are representative of the input genome; (c) identifying from a data structure, the at least one edit based on one or more reference edit patterns matching a pattern associated with the at least one edit, wherein the one or more reference edit patters are defined by segments of the multiple sequence reads of the output genome mapped onto the one or more reference sequences; (d) reporting the identified at least one edit in response to the request; (e) selecting the output genome based on the identified at least one edit; and (f) planting an organism consistent with the selected output genome in a growing space.

As will also be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the following operations: (a) receiving a request to identify at least one edit in an edited genome, the edited genome based on an input wild type genome and one or more edits to the input wild type genome, wherein one or more reference sequences are representative of the input wild type genome; (b) mapping sequence reads from the edited genome to the one or more reference sequences; (c) identifying from a data structure, the at least one edit of the one or more edits, based on one or more reference edit patterns matching a pattern associated with the at least one edit; and (d) reporting the identified at least one edit in response to the request.

The output information derived from methods or systems provided herein can be used to select subject organisms carrying a desired variation in its genome to additional breeding using one or more known methods in the art, e.g., pedigree breeding, recurrent selection, mass selection, and mutation breeding. Edits identified via methods and systems provided herein can be introgressed into different genetic backgrounds and selected via genotypic or phenotypic screening. For example, the genome may be selected based on the detected edits (i.e., desired edits) being confirmed by the systems and methods herein, whereby the selected genome may then form an organism such as, for example, a seed, etc. which is then planted in a growing space (e.g., a field, a greenhouse, plot, etc.).

Pedigree breeding starts with the crossing of two genotypes, such as a first plant comprising a selected edit and another plant lacking the selected edit. If the two original parents do not provide all the desired characteristics, other sources can be included in the breeding population. In the pedigree method, superior plants are self-pollinated and selected in successive filial generations. In the succeeding filial generations the heterozygous condition gives way to homogeneous varieties as a result of self-fertilization and selection. Further, edits that are not selected for, for example off-target edits are lost. Typically in the pedigree method of breeding, five or more successive filial generations of self-pollination and selection is practiced: F1 to F2; F2 to F3; F3 to F4; F4 to F5, etc. After a sufficient amount of inbreeding, successive filial generations will serve to increase seed of the developed variety. The developed variety may comprise homozygous alleles at about 95% or more of its loci.

In addition to being used to create a backcross conversion, backcrossing can also be used in combination with pedigree breeding. Backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with self-pollination and selection. For example, a first plant variety may be crossed with a second plant variety to produce a first generation progeny plant. The first generation progeny plant may then be backcrossed to one of its parent varieties to create a BC1 or BC2. Progenies are self-pollinated and selected so that the newly developed variety has many of the attributes of the recurrent parent and yet several of the desired attributes of the non-recurrent parent. This approach leverages the value and strengths of the recurrent parent for use in new plant varieties.

Recurrent selection is a method used in a plant breeding program to improve a population of plants. The method entails individual plants cross-pollinating with each other to form progeny. The progeny are grown and the progeny comprising a desired edit, such as an edit characterized according to the present methods and systems are selected by any number of selection methods, which include individual plant, half-sibling progeny, full-sibling progeny and self-pollinated progeny. The selected progeny are cross-pollinated with each other to form progeny for another population. This population is planted and again plants comprising a desired modification are selected to cross pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic line. A synthetic line is the resultant progeny formed by the intercrossing of several selected varieties.

Mass selection is another useful technique when used in conjunction with molecular marker enhanced selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Also, instead of self-pollination, directed pollination could be used as part of the breeding program.

Edits that are identified and/or characterized by the methods and systems provided herein can improve the agronomic characteristics of a plant. As used herein, the term “agronomic characteristics” refers to any agronomically important phenotype that can be measured. Non-limiting examples of agronomic characteristics include floral meristem size, floral meristem number, ear meristem size, shoot meristem size, root meristem size, tassel size, ear size, greenness, yield, growth rate, biomass, fresh weight at maturation, dry weight at maturation, number of mature seeds, fruit yield, seed yield, total plant nitrogen content, nitrogen use efficiency, resistance to lodging, plant height, root depth, root mass, seed oil content, seed protein content, seed free amino acid content, seed carbohydrate content, seed vitamin content, seed germination rate, seed germination speed, days until maturity, drought tolerance, salt tolerance, heat tolerance, cold tolerance, ultraviolet light tolerance, carbon dioxide tolerance, flood tolerance, nitrogen uptake, ear height, ear width, ear diameter, ear length, number of internodes, carbon assimilation rate, shade avoidance, shade tolerance, mass of pollen produced, number of pods, resistance to herbicide, resistance to insects and disease resistance.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. In addition, advantages and improvements that may be achieved with one or more example embodiments disclosed herein may provide all or none of the above mentioned advantages and improvements and still fall within the scope of the present disclosure.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on”, “engaged to”, “connected to”, “coupled to”, “associated with”, “in communication with”, or “included with” another element or layer, it may be directly on, engaged, connected or coupled to, or associated or in communication or included with the other feature, or intervening features may be present. As used herein, the term “and/or” and the phrase “at least one of” includes any and all combinations of one or more of the associated listed items.

Although the terms first, second, third, etc. may be used herein to describe various features, these features should not be limited by these terms. These terms may be only used to distinguish one feature from another. Terms such as “first”, “second”, and other numerical terms when used herein do not imply a sequence or order unless clearly indicated by the context. Thus, a first feature discussed herein could be termed a second feature without departing from the teachings of the example embodiments.

None of the elements recited in the claims are intended to be a means-plus-function element within the meaning of 34 U.S.C. § 112(f) unless an element is expressly recited using the phrase “means for,” or in the case of a method claim using the phrases “operation for” or “step for.”

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for identifying edits in output genomes, the method comprising:
receiving, by a computing device, a request to identify at least one edit in an output genome, the output genome based on an input genome and one or more edits to the input genome;
mapping, by the computing device, multiple sequence reads from the output genome onto one or more reference sequences, wherein the one or more reference sequences are representative of the input genome;
identifying, by the computing device, from a data structure, the at least one edit based on one or more reference edit patterns matching a pattern associated with the at least one edit, wherein the one or more reference edit patters are defined by segments of the multiple sequence reads of the output genome mapped onto the one or more reference sequences; and
reporting, by the computing device, the identified at least one edit in response to the request.

2. The method of claim 1, wherein the one or more reference edit patterns are further defined by locations and/or orientations of the segments of the multiple sequence reads of the output genome mapped onto the one or more reference sequences.

3. The method of claim 1, wherein each of the one or more reference sequences is representative of an input wild type genome.

4. The method of claim 1, wherein the at least one edit includes: a deletion edit, an inversion edit, a homolog fragment targeting (HFT) edit, and/or a trans-fragment targeting TFT edit; and
wherein the one or more reference edit patterns include one or a combination of simple edits including: a deletion edit, an inversion edit, a homolog fragment targeting (HFT) edit, and/or a trans-fragment targeting TFT edit.

5. The method of claim 1, further comprising determining whether the at least one edit is associated with only one target site of the output genome; and
wherein the at least one edit includes a simple edit in response to the at least one edit being associated with the only one target site.

6. The method of claim 5, wherein the target site is defined by guide RNA (gRNA).

7. The method of claim 1, further comprising:
determining whether the at least one edit is associated with more than one target site of the output genome; and
determining whether the at least one edit is associated with only one gene or locus;
wherein the at least one edit includes multiple edits selected from simple edits, inversion edits, and/or HFT edits, but not TFT edits, in response to the at least one edit being associated with more than one target site and being associated with only one gene or locus.

8. The method of claim 1, further comprising determining whether the at least one edit is associated with more than one gene or locus; and
wherein the one or more edit includes multiple edits selected from simple edits, inversion edits, HFT edits, and TFT edits, in response to the at least one edit being associated with more than one target site and being associated with more than one gene or locus.

9. The method of claim 1, further comprising searching, by the computing device, in the data structure, for the one or more reference edit patterns, in order to identify the at least one edit.

10. The method of claim 1, further comprising discarding ones of the sequence reads, prior to mapping the sequence reads onto the one or more reference sequences, based on the ones of the sequence reads being located apart from one or more target sites of the one or more reference sequences and/or or the output genome; and
wherein mapping the sequence reads includes mapping the non-discarded ones of the sequence reads onto the one or more reference sequences.

11. The method of claim 1, wherein the one or more reference edit patterns defined by the segments of the sequence reads mapped onto the one or more reference sequences includes:
one of the segments of a first one of the sequence reads spaced apart from a second one of the segments of the first one of the sequence reads, and oriented in an opposite direction relative to the second one of the segments of the first one of the sequence reads.

12. The method of claim 1, further comprising:
selecting the output genome based on the identified at least one edit; and
planting an organism consistent with the selected output genome in a growing space.

13. A non-transitory computer-readable storage medium including executable instructions for identifying edits in output genomes, which, when executed by at least one processor, cause the at least one processor to:
receive a request to identify at least one edit in an output genome, the output genome based on an input genome and one or more edits to the input genome;
map multiple sequence reads from the output genome onto one or more reference sequences, wherein the one or more reference sequences are representative of the input genome;
identify, from a data structure, the at least one edit based on one or more reference edit patterns matching a pattern associated with the at least one edit, wherein the one or more reference edit patterns are defined by segments of the multiple sequence reads of the output sequence mapped onto the one or more reference sequences; and
report the identified at least one edit in response to the request.

14. The non-transitory computer-readable storage medium of claim 13, wherein the one or more reference edit patterns are further defined by locations and/or orientations of the segments of the multiple sequence reads of the output genome mapped onto the one or more reference sequences.

15. The non-transitory computer-readable storage medium of claim 13, wherein the executable instructions, when executed by the at least one processor, further cause the at least one processor to, after mapping the multiple sequence reads from the output genome onto the one or more reference sequences and before identifying the at least one edit, keeping ones of the sequence reads having segments mapped onto different ones of the one or more reference sequences or onto two locations in opposite orientations of one of the one or more reference sequences.

16. The non-transitory computer-readable storage medium of claim 13, wherein the pattern associated with the at least one edit is defined by at least one location and/or at least one orientation of the segments of the sequence reads mapped onto the one or more reference sequences, relative to target sites on the one or more reference sequences.

17. The non-transitory computer-readable storage medium of claim 13, wherein the executable instructions, when executed by the at least one processor, further cause the at least one processor to discard ones of the sequence reads, prior to mapping the sequence reads onto the one or more reference sequences, based on the ones of the sequence reads being located apart from one or more target sites of the one or more reference sequences and/or or the output genome; and
wherein the executable instructions, when executed by the at least one processor, in order to map the multiple sequence reads, cause the at least one processor to map only non-discarded ones of the sequence reads onto the one or more reference sequences.

18. (canceled)

19. A method of identifying one or more edits in an output genome to which one or more edits have been made, the method comprising:
identifying, by a computing device, the one or more edits in the output genome based on at least one reference pattern identified by mapping multiple sequence reads of the output genome onto a reference sequence of an unedited version of the genome.

20. (canceled)

21. A method for identifying edits in genomes, the method comprising:
receiving, by a computing device, a request to identify at least one edit in an edited genome, the edited genome based on an input wild type genome and one or more edits to the input wild type genome, wherein one or more reference sequences are representative of the input wild type genome;
mapping, by the computing device, sequence reads from the edited genome to the one or more reference sequences;
identifying, by the computing device, from a data structure, the at least one edit of the one or more edits, based on one or more reference edit patterns matching a pattern associated with the at least one edit; and
reporting, by the computing device, the identified at least one edit in response to the request.

22. The method of claim 21, wherein the one or more reference edit patterns are defined by locations and/or orientations of the segments of the multiple sequence reads of the output genome mapped onto the one or more reference sequences.

23. (canceled)
